(19) United States
(12) Patent Application Publication
(10) Pub. No.: US 2021/0350906 A1
(43) Pub. Date: Nov. 11, 2021

(54) METHODS AND SYSTEMS FOR MEDICAL IMAGE MANAGEMENT

(71) Applicant: GE Precision Healthcare LLC, Milwaukee, WI (US)

(72) Inventor: Christopher Frank Janicki, Sleepy Hollow, IL (US)

(21) Appl. No.: 16/869,811

(22) Filed: May 8, 2020

(51) Int. Cl. G16H 30/20; G06F 3/0483; G06F 3/0484; G06F 3/0482; G16H 30/40; G06F 16/51; G06F 16/54

(52) U.S. Cl. CPC G16H 30/20; G06F 3/0483; G06F 3/04847; G06F 16/54; G16H 30/40; G06F 16/51; G06F 3/0482

(57) ABSTRACT

Various methods and systems are provided for management of images generated by medical imaging systems. In one embodiment, a method for a medical image management system comprises: acquiring a medical image dataset and displaying a timeline bar of the medical image dataset via a graphical user interface (GUI) of a display device; adjusting a position of a first key image indicator and a position of a second key image indicator along the timeline bar; generating an image subset from the medical image dataset based on the position of the first key image indicator and the position of the second key image indicator and storing the image subset for replay; and forming a visual indicator of the image subset within the GUI that when selected, displays the image subset.

### FIELD

Embodiments of the subject matter disclosed herein relate to medical imaging, and more particularly, to management of images generated by medical imaging systems.

### BACKGROUND

Clinicians, such as radiologists, often review large image datasets generated by medical imaging systems, such as ultrasound or computerized tomography (CT) imaging systems, to determine patient treatment and/or diagnosis. Image datasets generated by such medical imaging systems can include a large number of sequential image frames, and a number of image frames within an image dataset generated by a medical imaging system may correspond to a length of a scan of a patient and an image capture rate of the medical imaging system. Scans having a longer length or a higher image capture rate may generate an image dataset having a larger number of image frames. Depending on the type of scan being performed, the image dataset may include image frames showing a same anatomical feature of the patient at different times throughout the scan or different anatomical features at different times, such that different portions of the image dataset may provide different information about the anatomy of the patient being imaged.

### BRIEF DESCRIPTION

In one embodiment, a method for a medical image management system comprises: acquiring a medical image dataset and displaying a timeline bar of the medical image dataset via a graphical user interface (GUI) of a display device; adjusting a position of a first key image indicator and a position of a second key image indicator along the timeline bar; generating an image subset from the medical image dataset based on the position of the first key image indicator and the position of the second key image indicator and storing the image subset for replay; and forming a visual indicator of the image subset within the GUI that when selected, displays the image subset.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 is a block diagram schematically showing a medical image management system for a medical imaging system.

FIG. 2 shows a graphical user interface (GUI) of a medical image management system.

FIG. 3 shows the GUI of FIG. 2 with an image subset of a medical image dataset selected via key frames.

FIG. 4 shows the GUI of FIGS. 2-3 with a visual indicator of the image subset within the GUI including a customized label.

FIG. 5 shows the GUI of FIGS. 2-4 with additional visual indicators corresponding to additional image subsets.

FIG. 6 shows the GUI of FIGS. 2-5 with an additional visual indicator menu.

FIG. 7 is a flowchart illustrating an example method for managing medical images via a medical image management system.

### DETAILED DESCRIPTION

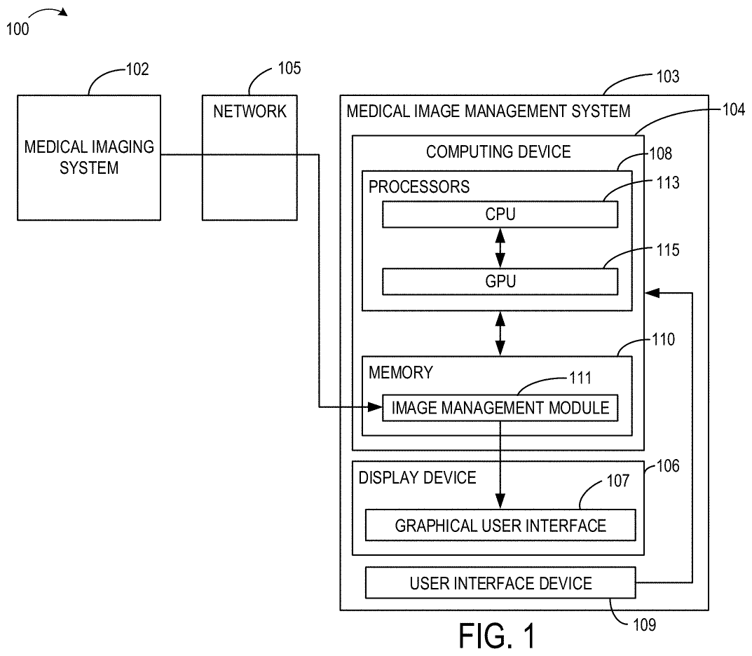

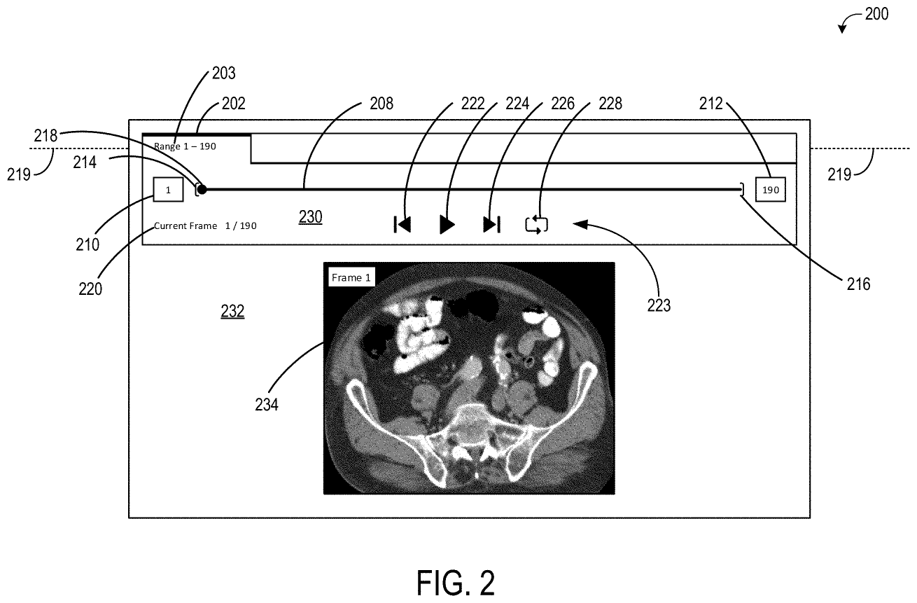

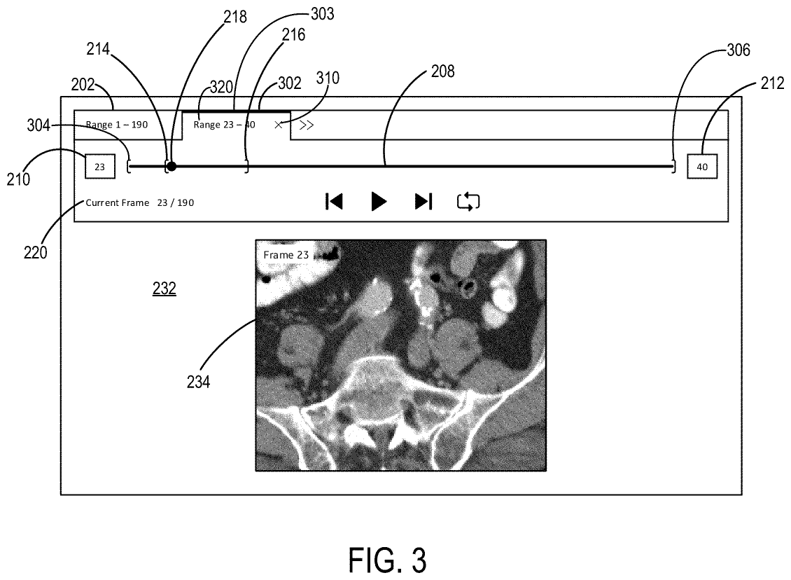

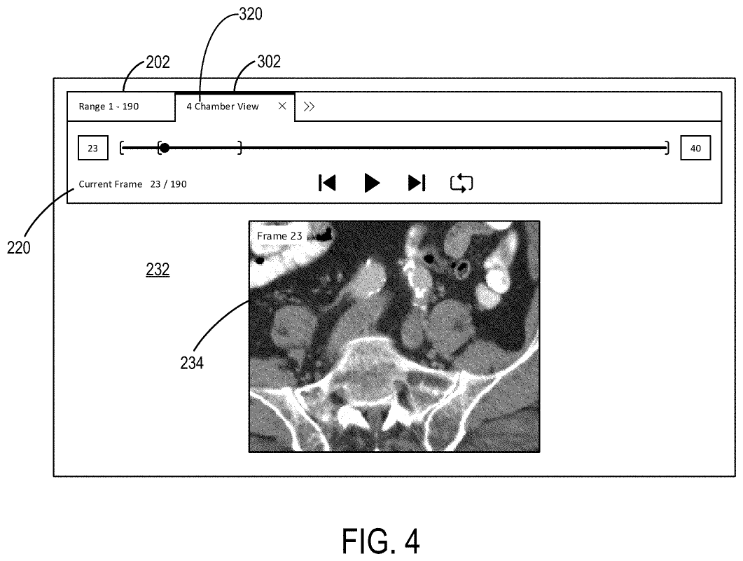

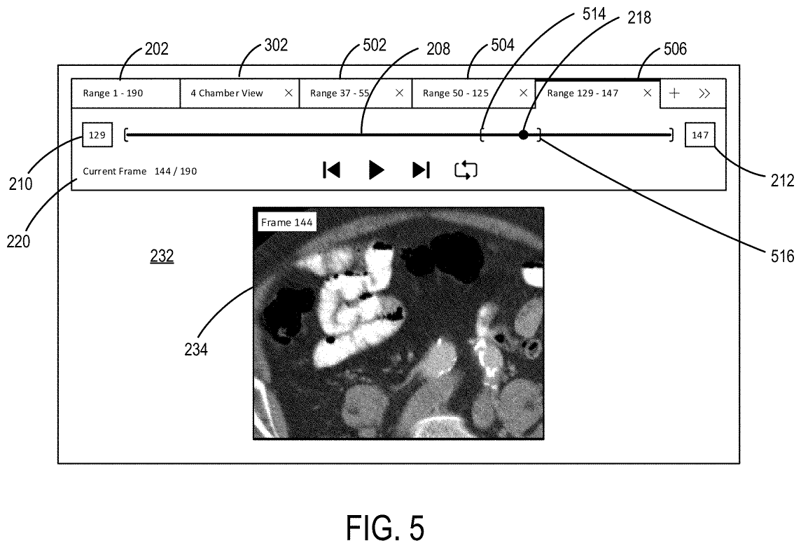

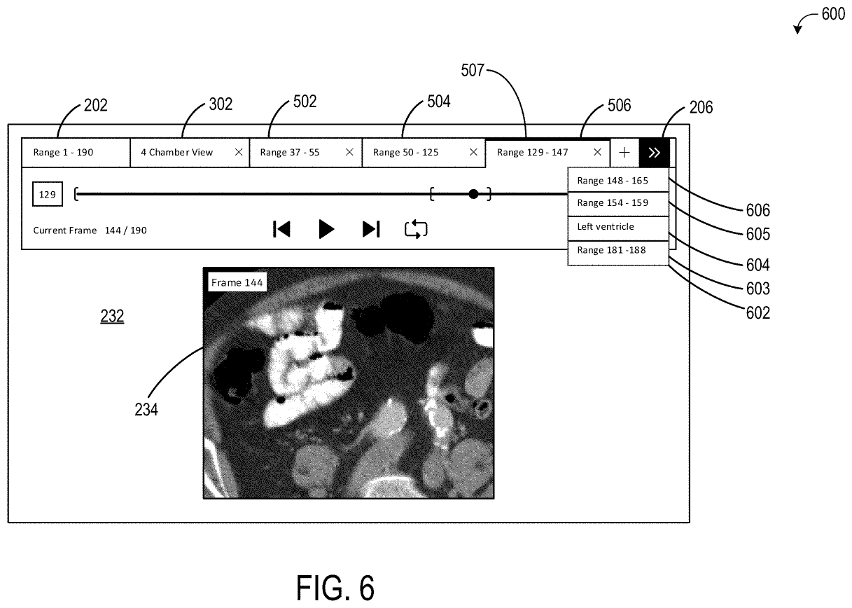

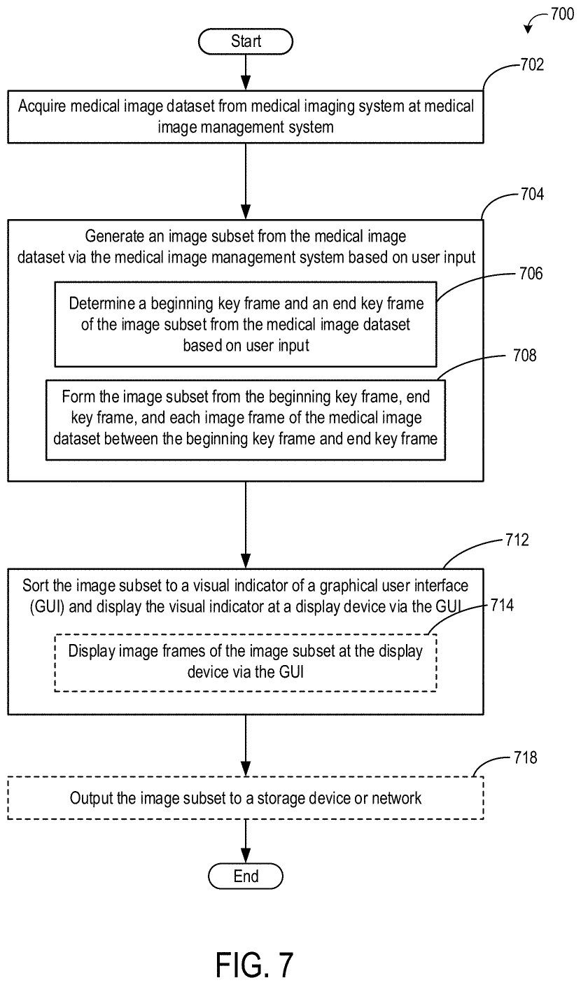

The following description relates to systems and methods for management of images generated by medical imaging systems. A medical image management system, such as the medical image management system shown by FIG. 1, is configured to acquire medical image datasets from one or more medical imaging devices and generate one or more image subsets, as illustrated by FIG. 7. The medical image management system displays the medical image dataset via a display device including a graphical user interface (GUI), such as the GUI shown by FIG. 2. The GUI includes a slider bar representing an entire range of the image dataset. A user of the medical image management system, such as a clinician, may define key frames from the medical image dataset via the slider bar. The medical image management system generates an image subset from the key frames and sorts the image subset to a visual indicator of the image subset within the GUI, such as the tab shown by FIG. 3. A label of the tab of the GUI may be customized by the user, as shown by FIG. 4. The medical image management system may generate additional image subsets having respective tabs responsive to user input, as shown by FIG. 5. During some conditions, one or more of the tabs may be displayed in an additional tab drop-down menu, as shown by FIG. 6. By configuring the medical image management system to generate one or more image subsets from the medical image dataset responsive to user input and sort the image subsets to respective tabs of the GUI, the user of the medical image management system may selectively view desired portions of the image data subset even during conditions in which one or more of the image subsets overlap each other. For example, a clinician reviewing time series image data of an image dataset (e.g., from a computerized tomography scan) may specify one or more image subsets corresponding to portions of the scan in which anatomical features of interest are more easily visible or identifiable.

The medical image management system disclosed herein provides specification and navigation of image subsets (e.g., image ranges or frame ranges) within a medical image dataset (e.g., a series of images). In some examples. some image subsets may overlap each other. As described herein, an overlapping image subset, or frame range, refers to an image subset which includes a key frame included within a frame range of another image subset (e.g., arranged between first and second key frames of another image subset). A nested image subset, or frame range, as described herein refers to an image subset that includes first and second key frames each included within a frame range of another image subset (e.g., first and second key frames of a first, nested image subset are included between first and second key frames of a second image subset).

An image subset, or frame range, may refer to a sequence of one or more images forming a subset of a medical image dataset, or larger image series. The images of the medical image dataset may be acquired sequentially in real time and may be stored to a storage device prior to acquisition by the medical image management system. In the following description, the terms “image” and “frame” may be used interchangeably.

Time series image data may include image sequences from video, or image sequences generated in real time by a medical imaging system, such as a radiological imaging system, ultrasound imaging system, computerized tomography (CT) imaging system, etc. For example, a medical image dataset may include 1000 images acquired sequentially. Responsive to a user input, the medical image management system may generate an image subset having a first key frame corresponding to frame 100 of the medical image dataset and a second key frame corresponding to frame 200 of the medical image dataset. The medical image management system may further generate a second image subset having a first key frame corresponding to frame 150 of the medical image dataset and a second key frame corresponding to frame 250 of the medical image dataset. In this configuration, the first image subset and second image subset overlap (e.g., a frame range of the first image subset at least partially overlaps a frame range of the second image subset). The medical image management system may further generate a third image subset having a first key frame corresponding to frame 120 of the medical image dataset and a second key frame corresponding to frame 180 of the medical image dataset. In this configuration, the third image subset is nested within the first image subset.

Overlapping or nested image subsets (e.g., frame ranges of the medical image dataset) may be represented via visual indicators such as tabs shown by the graphical user interface such that the frame ranges can be easily identified and selectively viewed. The medical image management system displays each new image subset in a separate tab of the graphical user interface. A user may select an image subset (which may be referred to herein as a frame range) of interest via input to the graphical display device, customize the label of the tab of the selected image subset, and view and navigate between various different image subsets generated from the medical image dataset by the medical image management system. The graphical user interface may also include interface elements for navigating image frames of the medical image dataset and image subsets, such as cine controls for playing, fast-forwarding, rewinding, etc. through image frames of the image subsets and/or medical image dataset.

The graphical user interface includes control elements that provide navigation through image subsets and display elements, such as tabs, that sort and display the image subsets. Each image subset includes a range of frames of the medical image dataset, with the range defined by a first key frame (e.g., a starting point in a sequence of images) and a second key frame (e.g., an end point the sequence of images). Two or more of the image subsets may be overlapping and/or nested image subsets.

Additionally, a user may customize the label of the respective tab associated with each image subset such that the user may more easily navigate between the image subsets by selecting the respective tab. Thus, a plurality of image subsets may be sorted to different tabs arranged along a top portion of the GUI, with one image subset associated with each tab, and where each image subset is labeled by the corresponding tab. The user may select a tab in order to view the corresponding image subset associated with the tab. The image subset of a selected tab is displayed in a display panel of the GUI, and cine controls are included for advancing through the image frames of the image subset, skipping to the beginning or end image frames of the image subset, cycling through images in a loop of images of the image subset, etc., as well as other controls as discussed further below.

Referring now to FIG. 1, a block diagram is shown that illustrates a system 100 for management of images generated by a medical imaging system. In particular, FIG. 1 shows medical imaging system 102 electronically coupled with medical image management system 103 (e.g., in electronic communication with medical image management system 103). Medical imaging system 102 is configured to generate a medical image dataset including time series image data (e.g., sequential images generated during a scan of a subject by the medical imaging system 102). Medical imaging system 102 may be configured to image a subject an imaging modality such as magnetic resonance imaging, nuclear medicine, ultrasound, elastography, photo acoustic imaging, tomography, eco-cardiology, etc. A medical image dataset including time series images of a subject may be generated by scanning the subject via the medical imaging system 102. As one non-limiting example, medical imaging system 102 may be an ultrasound imaging system configured for scanning the subject via one or more ultrasound devices (e.g., probes), where the medical image dataset generated by the medical imaging system includes a plurality of sequential ultrasound images of the subject acquired during the scan of the subject. As another non-limiting example, the medical imaging system 102 may be a computerized tomography (CT) imaging system configured to generate a medical image dataset including sequential images acquired during a CT scan of the subject (e.g., a brain of a patient).

The medical image dataset (which may be referred to herein as an image series dataset) is received by medical image management system 103. In particular, medical image management system 103 includes a computing device 104 having processors 108, memory 110, and display device 106. The medical image management system 103 receives the medical image dataset from the medical imaging system 102 and may store the medical image dataset in memory 110. In some examples, the medical image management system 103 may be included within the medical imaging system 102. For example, medical image management system 103 may be directly coupled to the medical imaging system 102, and computing device 104 may receive the medical image dataset store the medical image dataset in memory 110 as the medical image dataset is acquired by the medical imaging system 102 (e.g., via a scan of a subject by the medical imaging system 102). However, in the example shown by FIG. 1, the medical image management system 103 is separate from the medical imaging system 102 and is configured to receive the medical image dataset from the medical imaging system 102 via network 105. For example, medical image management system 103 may be arranged at a different location relative to the medical imaging system 102 (e.g., a different room, different building, etc.). The medical image management system 103 may acquire (e.g., retrieve) the medical image dataset from the medical imaging system 102 responsive to a user input (e.g., a user request to acquire the medical image dataset) in some examples, and in other examples, the medical imaging system 102 may automatically transmit the medical image dataset to the medical image management system 103 following completion of a scan of a subject by the medical imaging system 102. As such, the medical image management system 103 may be utilized to view the medical image dataset at a different location relative to the medical imaging system 102 and/or a different time relative to a time of the scan of the subject (e.g., after the scan of the subject has been completed). In some examples, the computing device 104 may be a computer such as a workstation or server, or a different type of computing device, such as a smart phone. However, in each example, the computing device 104 includes instructions stored in memory 110 (e.g., in image management module 111) for management of medical images of a medical image dataset, as described herein.

The computing device 104 includes processors 108. Processors 108 may include a central processing unit (CPU) 113 and a graphics processing unit (GPU) 115. In some examples, processors 108 may include additional processors not shown by FIG. 1 (e.g., one or more additional GPUs, etc.). The computing device 104 additionally includes memory 110. As discussed herein, memory includes non-transient computer readable medium in which programming instructions are stored. The computer readable medium includes computer readable storage (e.g., non-transitory storage) such as a flash memory, a read-only memory (ROM), a random-access memory (RAM), a cache, or other storage media in which information is stored for a duration (e.g. for extended or brief time periods). Computer memory of computer readable storage mediums as referenced herein may include volatile and non-volatile or removable and non-removable media for storage of electronic-formatted information such as computer readable program instructions or modules of computer readable program instructions, data, etc. that may be stand-alone or as part of a computing device. Examples of computer memory may include other medium which can be used to store the desired electronic format of information and which can be accessed by the processor or processors or at least a portion of the medical image management system 103.

Image management module 111 is stored within non-transitory memory of memory 110 of the medical image management system 103 and includes one or more image management algorithms for generating image subsets based on the medical image dataset acquired by the medical image management system 103 from medical imaging system 102. Processors 108 may execute image management instructions (e.g., an image management algorithm) stored by image management module 111 and may display an output of the image management algorithm at display device 106. For example, the output of the image management algorithm may include a graphical user interface (GUI) displayed at the display device 106. The processors 108 generate image subsets from the medical image dataset via the instructions stored by the image management module 111 based on user input and the medical image dataset acquired by the medical image management system 103 from the medical imaging system 102.

The display device 106 is configured to display image series data of the image subsets and/or medical image dataset (e.g., for viewing by a user of the medical image management system 103). In some examples, the display device 106 may be configured as a cathode ray tube (CRT) monitor, liquid crystal display (LCD) monitor, or light-emitting diode (LED) monitor. In other examples, such as examples in which the computing device 104 is a mobile (e.g., portable) computing device such as a smart phone, the display device 106 may be a screen of the mobile computing device. In some examples, the display device 106 may be a touchscreen configured to respond to a touch of the user in order to enable the user to interact with the GUI displayed by the display device 106.

The display device 106 is configured to display graphical user interface (GUI) 107, with the GUI 107 configured according to instructions stored in the image management module 111 and executed by the computing device 104 of medical image management system 103. A user may interact with the GUI 107 via a user interface device 109 in order to adjust the display of different image subsets of the medical image dataset which may be of interest for observation (e.g., for diagnosis and/or treatment of a patient). In some examples, the user interface device 109 may be a device such as a keyboard, mouse, trackball, etc. In other examples, the user interface device 109 may be d with the display device 106 as a touchscreen, such that the user may touch the touchscreen in order to interact with the GUI 112.

The GUI 112 may allow a user to view one or more image subsets generated by the medical image management system 103 via the image management module 111 stored in memory 110 of computing device 104. The user may interact with GUI 112 to select an image subset from a plurality of image subsets by selecting a visual indicator, such as a tab, associated with the image subset. A given image subset generated by the medical image management system 103 may be displayed while the tab associated with the image subset is selected, and the image subset may be hidden while the tab is not selected (e.g., during conditions in which a different tab of a different image subset is selected). The user may interact with controls (e.g., graphical buttons) of the GUI 112 via the user interface device 109 in order to view individual frames (e.g., images) of an image subset. For example, the user may provide input to the GUI 112 via the user interface device 109 to view frames of an image subset as an animation (e.g., sequentially, with each frame automatically transitioning to the next frame) or as individual frames (e.g., by moving from one frame to the next frame manually responsive to user input). Each image subset may be generated responsive to user input, where the user selects a beginning key frame (which may be referred to herein as a first key image) and an end key frame (which may be referred to herein as a second key image) of a desired image subset from the medical image dataset, and the medical image management system generates a corresponding image subset based on the beginning key frame and end key frame.

The starting frame (e.g., starting image) of the image subset generated by the medical image management system is the beginning key frame, and the end frame (e.g., end image) is the end key frame. In this configuration, the beginning key frame defines a beginning boundary of the image subset, and the end key frame defines an end boundary of the image subset. The image subset additionally includes each frame (e.g., each image) of the medical image dataset arranged sequentially between the beginning key frame and end key frame (e.g., as acquired sequentially by the medical imaging system 102 during a scan of a subject to form the medical image dataset).

The user may additionally customize a label displayed by the GUI 112 for each image subset generated by the medical image management system 103. As described further below, generating an image subset via the medical image management system 103 additionally generates a visual indicator, such as a tab, associated with the image subset, with the tab including a label indicating a frame number of the beginning key frame and a frame number of the end key frame of the image subset. The GUI 112 may display multiple tabs, and the user may interact with a given tab (e.g., select a given tab) in order to display the image subset associated with the given tab. The user may customize the label of the given tab as desired in order to indicate the anatomy shown in the image frames of the image subset, a scanning time associated with the image frames of the image subset, or other desired information associated with the image subset. One or more of the image subsets may be in an overlapping or nested configuration relative to other image subsets. However, by sorting each image subset to a respective tab, the image subsets (including overlapping and/or nested image subsets) may be more easily navigated by the user of the GUI 112. In this way, user productivity may be increased and image subsets showing anatomical features of interest may be more easily organized for record keeping, storage, patient diagnosis and/or treatment, and/or repeated viewing.

Referring now to FIG. 2, a graphical user interface (GUI) 200 is shown. The GUI 200 may be similar to, or the same as, GUI 107 of medical image management system 103 shown by FIG. 1 and described above. In particular, GUI 200 is a GUI of a medical image management system, such as the medical image management system 103 described above with reference to FIG. 1.

FIG. 2 shows the GUI 200 in a configuration in which the medical image management system including the GUI 200 has acquired a medical image dataset and loaded the medical image dataset into the GUI 200 for display, but no image subsets have been generated by the medical image management system based on the medical image dataset. Graphical user interface 200 includes a layout displaying visual indicators of image subsets. In the example shown, the visual indicators are tabs. In this configuration, graphical user interface 200 includes a single, main tab 202, where the main tab 202 is associated with the entire frame range of the medical image dataset. In particular, in the configuration shown by FIG. 2, the image frames of the entire medical image dataset may be viewed in display panel 232 of GUI 200. For example, although the display panel 232 of GUI 200 shows only a single image of the medical image dataset as described further below, a user of the GUI 200 (e.g., a clinician) may interact with the GUI 200 in order to advance the frame displayed in the display panel 232 to a next sequential frame included by the medical image dataset, or reverse the frame displayed in the display panel 232 to a previous sequential frame included by the medical image dataset, as described below.

The GUI 200 includes a control panel 230, which may include a plurality of control elements 223 that a user (e.g., a clinician) may interact with to advance or reverse the image displayed by display panel 232 through a range of sequential images. For example, in the configuration shown by FIG. 2, the main tab 202 is associated with an entire frame range of a medical image dataset. A single frame of the medical image dataset may be displayed in the display panel 232 (e.g., at viewport 234). The user may use the control elements 223 of the control panel 230 to advance the frame displayed in the display panel 232 to the next sequential frame included by the medical image dataset, or reverse the frame displayed in the display panel 232 to the previous sequential frame included by the medical image dataset. The user may further use the control elements 223 to view the medical image dataset as a replayable animation at the location of viewport 234 (e.g., an animation that may be played more than once). In some examples, the user may further advance a playback speed of the animation and/or reverse a direction of the animation using the control elements 223 of control panel 230. In the example shown by FIG. 2, the display panel 232 is arranged vertically below the control panel 230, with the viewport 234 displayed centrally within the display panel 232. However, in other examples, the display panel 232 may be arranged vertically above the control panel 230, to a side of the control panel 230, etc.

The main tab 202 includes a label 203 indicating an entire frame range of the medical image dataset. For example, a medical image dataset may include a total of 190 frames, and during conditions in which the medical image dataset is loaded to the GUI 200 for viewing by a user at display panel 232, the label 203 correspondingly indicates that frames 1 through 190 are viewable at the display panel 232 (e.g., viewable one frame at a time, or viewable as a replayable animation). In the example shown by FIG. 2, the medical image dataset includes a total of 190 frames, and the label 203 of the main tab 202 correspondingly displays “Range 1-190” to indicate that the control panel 230 may be interacted with by the user to navigate the entire medical image dataset while the main tab 202 is selected. During conditions in which one or more image subsets are generated from the medical image dataset, additional tabs are displayed at a same vertical position of the GUI 200 as main tab 202, as indicated by axis 219. A tab of a first image subset may be displayed adjacent to the main tab 202 along axis 219, a tab of a second image subset may be displayed adjacent to the first tab along axis 219, and so forth. As each image subset is generated, the most recently generated image subset may be automatically selected by the GUI 200 (e.g., selected responsive to the generation of the image subset). Selection of a tab removes the image frame currently displayed at the viewport 234 of display panel 232 (e.g., clears the display panel) and replaces the image frame at the viewport 234 with the first frame (e.g., beginning key frame) of the image subset associated with the selected tab. Additional tabs are described in further detail below.

GUI 200 may include an image track 208 (which may be referred to herein as a slider bar or timeline bar), displayed in FIG. 2 as a horizontal line representing the entire duration of a loaded time series (e.g., the entire frame range of the medical image dataset in the configuration shown by FIG. 2). The image track 208 includes a first bracket 214 (which may be referred to herein as a first marker) arranged at a first end of the image track 208 to indicate the first frame of the medical image dataset (e.g., frame 1) and a second bracket 216 (which may be referred to herein as a second marker) arranged at a second end of the image track 208 to indicate the last frame of the medical image dataset (e.g., frame 190). The individual frames may be numbered within the medical image dataset, such that different locations along image track 208 correspond to different frames included within the medical image dataset. Frame reference indicator 218 is arranged along image track 208 to indicate the sequential order of the frame currently displayed at display panel 232 (e.g., at viewport 234) relative to the other frames included by the medical image dataset. In the example shown, the frame reference indicator 218 is a circle centered vertically along the image track 208. However, in other examples, frame reference indicator 218 may have a different shape (e.g., frame reference indicator 218 may be shaped as a rectangle, triangle, etc.). Frame reference indicator 218 may be selected by the user via an input device such as a mouse, and moved laterally (e.g., horizontally) along the image track 208 in order to navigate between the individual images comprised by the entire dataset (e.g., display the different images one at a time at the position of viewport 234 at display panel 232). The current position of the frame reference indicator 218 may correspond to a “current frame” shown at viewport 234. For example, a user may select frame reference indicator 218 with a mouse and slide it down image track 208 to a new frame reference position, whereupon the mouse button is released. In some embodiments, releasing the mouse button may trigger the display of the frame corresponding to the current position of frame reference indicator 218 at viewport 234. Alternatively, the frames (e.g., images) displayed at viewport 234 may update in real time as the user moves the frame reference indicator 218 to the left or to the right on image track 208.

First frame number indicator 210 and second frame number indicator 212, positioned at the left and right sides of image track 208, respectively, may indicate the frame number of the frames that define the beginning and end of the frame range shown on image track 208. In particular, first frame number indicator 210 displays the frame number of the first frame of the medical image dataset in FIG. 2, and second frame number indicator 212 displays the frame number of the last frame of the medical image dataset in FIG. 2 (e.g., due to the main tab 202 being the currently selected tab in the configuration shown by FIG. 2). During conditions in which the main tab 202 is displayed by the GUI 200 and no other tabs have been generated (e.g., during conditions in which no image subsets have been generated), the first frame number indicator 210 is arranged at the left side position representing the first (e.g., beginning) frame of the medical image dataset, and the second frame number indicator 212 is arranged at the right side position representing the last frame of the medical image dataset. The frame number of the currently selected frame, as indicated by the frame reference indicator 218, may be displayed textually at frame location identifier 220 based on where frame reference indicator 218 is positioned along image track 208. The image corresponding to the currently selected frame is displayed at viewport 234 in display panel 232.

GUI includes cine controls for progressing through the series of images, shown by control elements 223. The control elements 223 may be maintained in the position shown by FIG. 2 even during conditions in which a tab other than the main tab 202 is selected. Skip-to-beginning button 222 may be selected by the user via an input device such as a mouse (e.g., similar to the user interface device 109 described above with reference to FIG. 1) in order to adjust the currently selected frame to be the first frame of the image series currently being viewed (e.g., the first frame of the medical image dataset in FIG. 2, corresponding to the position of the frame reference indicator 218 at the left end of the image track 208). Skip-to-end button 226 may be selected by the user via the input device in order to adjust the currently selected frame to be the last frame of the image series currently being viewed (e.g., the last frame of the medical image dataset in FIG. 2, corresponding to the position of the frame reference indicator 218 at the right end of the image track 208). Play button 224 may be selected by the user to display the images of the currently selected image series in a replayable, sequential animation (e.g., playback the images of the currently selected image series from the current frame position in real time, such as at the rate at which they were captured by the medical imaging system). After the play button 224 is selected and while the images of the currently selected image series are displayed in the sequential animation, play button 224 may be replaced by a stop button (for example, identified as a black rectangle). If the user selects the stop button, the sequential animation stops and the image associated with the current position of the frame reference indicator 218 along the image track 208 is displayed at display panel 232. Once the stop button is selected, the stop button reverts back to play button 224. For example, a user who selects the play button when the frame reference indicator 218 is displayed at the first position in the image track as shown in FIG. 2, corresponding to the first image in an image series comprising 190 frames (e.g., the medical image dataset), may see the frames displayed at viewport 234 in real time as the frame reference indicator advances along the image track 208. In some embodiments, a user may be able to set the speed, direction, or other variables of the play button and functionality.

Through the features described above, GUI 200 may display time series image data comprising a range of frames, where the total number of frames is represented graphically along the image track, and whereby the user can select and move the frame reference indicator freely along the image track to navigate manually between individual images (e.g., display individual frames as desired at the display panel 232). Further, the user may advance the display of the images through the sequence of the images in real time or manually via the cine control elements 223 (e.g., the play button 224, skip-to-end button 226, skip-to-beginning button 222, etc.) while viewing the individual images in the display panel of the GUI 200. An indication of the total number of images and the image currently being viewed is indicated by both of the visual position of the frame reference indicator 218 along the image track 208 relative to the opposing ends of the image track, and by the frame location identifier 220.

The user may interact with the GUI 200 in order to generate one or more image subsets from the medical image dataset, as described further below. In particular, the user may select first bracket 214 with a user interface device (e.g., a mouse, as described above with reference to user interface device 109 shown by FIG. 1), move (e.g., slide) the first bracket 214 from the first end (e.g., the left end) of the image track 208 toward the second end (e.g., the right end) of the image track 208, and release the first bracket 214 at a desired location along the image track 208 in order to define the beginning key frame of a new image subset. Additionally or alternatively, the user may select second bracket 216 with the user interface device, move (e.g., slide) the second bracket 216 from the second end (e.g., the right end) of the image track 208 toward the first end (e.g., the left end) of the image track 208, and release the second bracket 216 at a desired location along the image track 208 in order to define the end key frame of the new image subset. As the user selects and moves the first bracket 214 to the right, an additional bracket may be maintained at the starting location of the first bracket 214 (e.g., at the first end of the image track 208), an as the user selects and moves the second bracket 216 to the left, an additional bracket may be maintained at the starting location of the second bracket 216 (e.g., at the second end of the image track 208). An example is shown by FIG. 3 and described below. By maintaining additional brackets at the opposing ends of the image track 208, further image subsets may be generated (e.g., by moving the additional brackets in a similar way, with the additional bracket at the first end being moved to the right, and/or the additional bracket at the second end being moved to the left). Thus, after generating a new image subset, the first bracket 214 and second bracket 216 may be arranged at start and end positions of the image subset, while an additional bracket remains at the original position of first bracket 214 (e.g., at the left end of image track 208) and an additional bracket remains at the original position of the second bracket 216 (e.g., at the right end of the image track 208), as described below and shown by FIG. 3.

Referring to FIG. 3, GUI 200 is shown in a configuration in which the user has generated an image subset via the medical image management system by moving the first bracket 214 and second bracket 216 to desired positions along the image track 208. As a result, as described above, bracket 304 is maintained at the original position of first bracket 214 (e.g., the position of the first bracket 214 prior to being moved by the user), and bracket 306 is maintained at the original position of second bracket 216 (e.g., the position of second bracket 216 prior to being moved by the user). Frame reference indicator 218 is displayed at the beginning of the image subset, at a position corresponding to a current frame of the image subset (e.g., frame number 23 as shown in frame location identifier 220), where the current frame is displayed at the display panel 232 (e.g., at the position of viewport 234). The image subset is nested with respect to the medical image dataset (e.g., the first bracket 214 and second bracket 216 defining the bounds of the image subset are each arranged between the bracket 304 and the bracket 306 indicating the bounds of the medical image dataset). In the example shown by FIG. 3, the beginning key frame of the image subset as indicated by first bracket 214 is frame number 23 of the medical image dataset, and the end key frame of the image subset as indicated by second bracket 216 is frame number 40 of the medical image dataset. The frame number of the beginning key frame (e.g., frame number 23) is displayed by first frame number indicator 210, and the frame number of the end key frame (e.g., frame number 40) is displayed by second frame number indicator 212.

While the user selects and moves the first bracket 214 and the second bracket 216 to generate the image subset shown by FIG. 3, a tab 302 associated with the image subset is automatically generated and selected by the medical image management system. During conditions in which the tab 302 is selected, the user may view the image frames of the image subset at the display panel 232 as described above (e.g., control playback of a replayable animation of the image frames, etc.). The user may select between the tab 302 and the main tab 202 in order to select which range of images of the medical image dataset to view at display panel 232. In particular, during conditions in which the main tab 202 is selected, the entire medical image dataset may be viewed at the display panel 232 (e.g., viewed as an animation or individual images at the position of viewport 234). During conditions in which the tab 302 is selected, only the image subset is viewable at the display panel 232, where the image subset includes only the beginning key frame, the end key frame, and the image frames of the medical image dataset arranged sequentially between the beginning key frame and the end key frame (with the position of the beginning key frame within the medical image dataset represented by the position of first bracket 214 along the image track 208, and with the position of the end key frame within the medical image dataset represented by the position of second bracket 216 along the image track 208, as described above). The selection of the tab 302 may be indicated by highlight portion 303, in some examples. During conditions in which the tab 302 is selected, highlight portion 303 may include a fill (e.g., a color fill configured to contrast with surrounding portions of the GUI), and during conditions in which the tab 302 is not selected, highlight portion may not include the fill. In other examples, the selection of tab 302 may be indicated in a different way (e.g., via virtual shadowing, or other graphical effects or icons, applied to the tab 302).

Each time the user interacts with the GUI 200 to generate an additional image subset via the medical image management system, an additional tab associated with the additional image subset is generated and displayed by the GUI 200. The user may select the desired tab to display the image subset associated with the selected tab at the display panel 232. The user may generate a plurality of image subsets from the medical image dataset, and the image subsets are sorted by the associated tabs (e.g., with a first image subset being displayed when a first tab is selected, a second image subset being displayed when a second tab is selected, etc.). In this configuration, the user may more easily navigate various image subsets and/or generate additional image subsets by selecting, moving, and releasing brackets representing image subset key frames on the horizontal image track 208. Further, for a given image subset, the user may adjust the bounds of the image subset (e.g., the frame range of the image subset relative to the medical image dataset) by adjusting the position of the brackets indicating the position of the beginning key frame and end key frame of the image subset. For example, the user may adjust the position of the first bracket 214 in order to increase or decrease the frame range of the image subset associated with tab 302. In some examples, the tab associated with an image subset may be generated at the moment that the user releases a mouse button after moving a bracket to a desired position along image track 208 to indicate a position of a beginning key frame or an end key frame of the image subset. In other examples, the tab associated with the image subset may not be generated until the user has defined both of the beginning key frame and the end key frame of the image subset (e.g., by moving brackets to indicate the position of both of the desired beginning key frame and the desired end key frame along the image track 208). In yet other examples, GUI 200 may include a confirmation button such that the user may generate an additional tab manually using the confirmation button. For example, the GUI 200 may be configured such that the brackets indicating the beginning key frame and end key frame may be repositioned until the confirmation button is selected (e.g., without generating the image subset and associated tab), and responsive to the selection of the confirmation button, the image subset and associated tab are generated by the medical image management system. As another example, the GUI 200 may include a menu including an input for a frame number of a desired beginning key frame, an input for a frame number of a desired end key frame, and the confirmation button, where inputting the frame numbers and selecting the confirmation button may generate the image subset and the associated tab.

Each tab, such as tab 302, may include a respective tab deletion button, such as tab deletion button 310. The user may interact with (e.g., click on via a mouse, touch via a touch screen, etc.) a tab deletion button to delete a tab associated with a corresponding image subset. Deleting the tab via the tab deletion button additionally may clear the image subset (e.g., remove the image subset from the GUI). In the example shown by FIG. 3, the tab 302 includes tab deletion button 310. During conditions in which the user selects the tab deletion button 310, the tab 302 is cleared (e.g., removed) from the GUI 200, and as a result, the image subset associated with the tab 302 is also cleared from the GUI 200. In particular, although the image subset is removed, the frames of the medical image dataset that formed the image subset are not removed (e.g., the medical image dataset is not altered as a result of the user clearing the image subset from the GUI 200 via the tab deletion button 310). As one example, GUI 200 may include three different tabs associated with respective image subsets, with a first tab (which may be referred to as a left tab) being adjacent to the main tab 202, a second tab (which may be referred to as a center tab) being adjacent to the first tab, and a third tab (which may be referred to as a right tab) being adjacent to the second tab. The GUI 200 may be in a configuration in which the right tab is selected, and the image subset associated with the right tab is displayed at the display panel 232. During conditions in which the user selects the tab deletion button of the right tab, the right tab is closed (e.g., removed from the GUI 200) and tab adjacent to the right tab (e.g., the center tab) may be automatically selected such that the image subset associated with the center tab replaces the image subset associated with the right tab at the display panel 232. The main tab 202 may not include a tab deletion button or the tab deletion button may be disabled in some examples, such as during conditions in which the main tab 202 is the only tab included by the GUI 200 (e.g., conditions in which the medical image management system has not generated an image subset and associated tab responsive to user input).

During conditions in which the GUI includes more than a threshold number of tabs (e.g., 5 tabs, 6 tabs, etc.), an additional tab indicator 206 may also be displayed, which when selected may open an additional tab menu (e.g., a drop-down menu) of tabs that are generated after the threshold number of tabs has been reached. For example, during conditions in which the threshold number of tabs is 5 tabs but the medical image management system has generated a total of 8 tabs responsive to user input (e.g., corresponding to 8 image subsets), the first, second, third, fourth, and fifth tabs may be displayed along the top portion of the GUI 200 (e.g., along axis 219 shown by FIG. 2), while the sixth, seventh, and eighth tabs may be displayed at the additional tab menu responsive to user input to the additional tab indicator 206 (e.g., selection of the additional tab indicator 206 by the user via the user interface device). An example additional tab menu is shown by FIG. 6 and described further below.

Referring to FIG. 4, GUI 200 is shown in a configuration in which the label 320 of the tab 302 has been customized according to user input. In one example, the user may customize the label of a tab, such as the label 320 of tab 302, by interacting with the label via the user interface device (e.g., double-clicking the label via a mouse, pressing a button of the user interface device configured to select the label for customization, etc.). Customizing the label may include adjusting a text string displayed by the label. In the example shown by FIG. 4, the label has been customized to include the text string “4 chamber view” instead of the text string “Range 23-40” such that the user may more quickly identify the tab associated with the image subset that includes images showing the 4 chamber view (e.g., a view of a heart of a patient). The user may customize the labels of various tabs in order to more easily identify which tabs are associated with image subsets that show anatomical features of interest (e.g., with each image subset corresponding to a range of image frames included by the medical image dataset), etc. The customization of the labels may increase an ease of use of the GUI 200 and reduce a navigation time of the image subsets, increasing user productivity.

Referring to FIG. 5, the GUI 200 is shown in a configuration in which a plurality of image subsets (and their associated tabs) have been generated by the medical image management system. In particular, GUI 200 is shown including the main tab 202 corresponding to the entire medical image dataset and tab 302 corresponding to the first image subset. GUI 200 is shown additionally including tab 502 corresponding to a second image subset generated by the medical image management system responsive to user input, tab 504 corresponding to a third image subset, and tab 506 corresponding to a fourth image subset. In the views shown by FIGS. 5-6, the tab 506 corresponding to the fourth image subset is selected (e.g., as indicated by the fill shown at highlight portion 507 of the tab 506).

The image subsets associated with each of the tabs may correspond to different frame ranges of the medical image dataset. In some examples, two of the image subsets may be nested and/or overlap each other, as described above. Tab 502 corresponds to an image subset having a beginning key frame at frame 37 of the medical image dataset and an end key frame at frame 55 of the medical image dataset; tab 504 corresponds to an image subset having a beginning key frame at frame 50 of the medical image dataset and an end key frame at frame 125 of the medical image dataset; and tab 506 corresponds to an image subset having a beginning key frame at frame 129 of the medical image dataset and an end key frame at frame 147 of the medical image dataset. The tab 506 is selected such that the image subset having the beginning key frame at frame 129 of the medical image dataset and the end key frame at frame 147 of the medical image dataset is displayed at the display panel 232 of the GUI 200. Additionally, the image track 208 shows first bracket 514 arranged at the position corresponding to frame number 129 of the medical image dataset along image track 208 (as additionally indicated by first frame number indicator 210) and second bracket 516 arranged at the position corresponding to frame number 147 of the medical image dataset along image track 208 (as additionally indicated by second frame number indicator 212). The frame number of the currently displayed frame of the image subset associated with tab 506 (e.g., frame 144) is indicated at frame location identifier 220. Further, the frame number of the currently displayed frame is indicated graphically by the position of frame reference indicator 218 along the image track 208 (e.g., in relation to the opposing ends of the image track indicating the start and end of the medical image dataset).

Referring to FIG. 6, the GUI 200 is shown in a configuration in which the GUI 200 includes the tabs described above with reference to FIG. 5, and additionally includes tabs displayed by additional tab menu 602. For example, in transitioning from the configuration shown by FIG. 5 to the configuration shown by FIG. 6 (e.g., following the configuration shown by FIG. 5 and prior to the configuration shown by FIG. 6), the user has interacted with the GUI 200 to cause the medical image management system to generate additional image subsets associated with tab 603, tab 604, tab 605, and tab 606 shown by FIG. 6. As described above, during conditions in which the GUI includes more than a threshold number of tabs (e.g., 5 tabs, 6 tabs, etc.), additional tab indicator 206 may also be displayed, which when selected may open the additional tab menu (e.g., drop-down menu) of tabs that are generated after the threshold number of tabs has been reached. In the example shown by FIG. 6, the threshold number of tabs is 5 tabs (including the main tab 202), and the image subsets corresponding to tab 603, tab 604, tab 605, and tab 606 are in addition to the threshold number of tabs. For example, tab 603 may correspond to a sixth image subset, tab 604 may correspond to a seventh image subset, tab 605 may correspond to an eighth image subset, and tab 606 may correspond to a ninth image subset. As a result, the tab 603, tab 604, tab 605, and tab 606 are normally hidden (e.g., when one of the main tab 202, tab 302, tab 502, tab 504, or tab 506 are selected), and during conditions in which the additional tab indicator 206 is selected, the drop-down menu 602 including the tab 603, tab 604, tab 605, and tab 606 is displayed. The label of one or more of the tabs within the drop-down menu 602 or other tabs shown by the GUI may be customized, similar to the customization of label 320 of tab 302 as described above. The user may visually inspect, navigate between, and select from the various image subsets using the additional tab indicator 206 and drop-down menu 602 as described above.

As described above, some of the image subsets may be in a nested and/or overlapping arrangement relative to each other. For example, the image subset associated with tab 605 is in a nested arrangement with the image subset associated with tab 606 (e.g., due to the image subset associated with tab 606 including the range of image frames between frame 148 of the medical image dataset and frame 165 of the medical image dataset, and the image subset associated with tab 605 including the range of image frames between frame 154 of the medical image dataset and frame 159 of the medical image dataset). In particular, each of the image frames included by the image subset associated with the tab 605 are also included by the image subset associated with the tab 606. The image subset associated with tab 606 includes a larger, first range of image frames of the medical image dataset, the image subset associated with tab 605 includes a smaller, second range of image frames of the medical image dataset, and the second range of image frames is nested within the first range of image frames as described above (e.g., the second range is included entirely within the first range, such that the first range includes each of the image frames of the second range and further includes additional image frames outside of the second range). Further, the image subset associated with tab 504 is in an overlapping arrangement with the image subset associated with the tab 502. In particular, the image subset associated with tab 504 and the image subset associated with tab 502 each include the image frames 50-55 of the medical image dataset. In this configuration, the image frames 50-55 of the medical image dataset may be viewed by the user during conditions in which either of the tab 502 or the tab 504 is selected. However, the image subset associated with tab 504 does not include image frames 37-49 of the medical image dataset (which are included by the image subset associated with tab 502), and the image subset associated with tab 502 does not include the image frames 56-125 of the medical image dataset (which are included by the image subset associated with tab 504).

In some examples, as shown by FIG. 6, the GUI 200 may display brackets associated with tabs that are not selected in order to indicate which image frames of the medical image dataset are included by each image subset without displaying the image frames at the display panel 232. However, the brackets associated with the tabs that are not selected have a different appearance relative to the brackets associated with the tab that is selected. For example, in the configuration shown by FIG. 6, the tab 506 is selected and the tab 302 is not selected. As a result, the first bracket 514 and the second bracket 516 associated with the image subset of the tab 506 are each shown in solid lines, while the first bracket 214 and the second bracket 216 associated with the image subset of the tab 302 are shown in dashed lines. In some examples, the GUI 200 may display brackets associated with each tab that is not selected, with the brackets shown having the alternate appearance (e.g., in dashed lines) relative to the brackets associated with the image subset of the selected tab. Although the first bracket 214 and second bracket 216 are shown in dashed lines in FIG. 6, in some examples the first bracket 214 and second bracket 216 may be indicated with a different appearance (e.g., a different color, opacity, shape, etc. relative to first bracket 214 and second bracket 216). Further, the brackets associated with tabs that are not selected may be shown with different appearances for each different tab. For example, during conditions in which the tab 302 and the tab 502 are each not selected, the first bracket 214 and second bracket 216 associated with the image subset of the tab 302 may be displayed with a first color, and the brackets associated with the image subset of the tab 502 may be shown in a different, second color at their corresponding positions along the image track 208. Each tab may include a color corresponding to the color of the brackets associated with the tab. In this way, the user may more easily and quickly identify which tab is associated with which image subset via the appearance of the brackets along the image track 208.

Referring to FIG. 7, a flowchart illustrating a method 700 for managing medical images via a medical image management system. The medical image management system described with reference to method 700 may be the medical image management system 103 shown by FIG. 1 and described above. Instructions for carrying out method 700 and the rest of the methods included herein may be executed by a computing device (e.g., computing device 104 shown by FIG. 1 and described above) based on instructions stored on a memory of the computing device (e.g., stored on image management module 111 of memory 110 shown by FIG. 1 and described above) and in conjunction with input provided to the computing device (e.g., via a user interface device, such as user interface device 109 shown by FIG. 1 and described above) by a user of the computing device (e.g., a clinician).

At 702, a medical image dataset is acquired from a medical imaging system at the medical image management system. The medical imaging system may be the medical imaging system 102 described above with reference to FIG. 1. The medical image dataset includes time series image data as described above (e.g., a plurality of image frames acquired throughout a scan of a subject by the imaging system). Acquiring the medical image dataset at the medical image management system may include transmitting the medical image dataset to the medical image management system via a network, such as the network 105 shown by FIG. 1 and described above. In other examples (e.g., examples in which the medical image management system is included within, or directly electronically coupled to, the medical imaging system), the medical image dataset may be acquired by the medical image management system by storing the medical image dataset to the memory of the medical image management system while the medical image dataset is generated by the medical imaging system (e.g., during a scan of a patient).

At 704, an image subset is generated from the medical image dataset via the medical image management system based on user input. For example, as described above, the medical image management system may display the medical image dataset at a display device via a graphical user interface (GUI), and the user of the medical image management system may interact with the GUI (e.g., input selections to the GUI via the user interface device) in order to define parameters of the image subset generated by the medical image management system. The display device may be similar to (or the same as) the display device 106 shown by FIG. 1 and described above, and the GUI may be similar to (or the same as) the GUI shown by FIGS. 2-6 and described above. The user input may include selecting and/or moving one or more brackets or other indicators of the GUI in order to define a desired frame range of the medical image dataset to be used for generation of the image subset, as described below.

Generating the image subset may include, at 706, determining a beginning key frame and an end key frame of the image subset from the medical image dataset based on user input. For example, the user may input, via the user interface device (e.g., similar to user interface device 109 described above with reference to FIG. 1), a selection of the beginning key frame and end key frame within the GUI to define the boundaries of the image subset.

As one example, selecting the beginning key frame may include selecting and moving a first bracket (e.g., first bracket 214 described above) from a first end of a slider bar (e.g., image track 208 described above) to a desired beginning image frame of the image subset, where different locations along the slider bar correspond to different image frames of the medical image dataset. As the user moves the first bracket along the slider bar, a frame number corresponding to the position of the first bracket along the slider bar may be displayed by the GUI for each of the different locations, and the user may release the first bracket (e.g., deselect the first bracket by releasing a button of the user interface device, such as a mouse button) at the desired location in order to define the beginning key frame (e.g., define the image frame of the medical image dataset that forms the beginning key frame of the image subset). The beginning key frame is an image frame that is selected from the plurality of image frames of the medical image dataset (e.g., the total amount of image frames of the medical image dataset) as described above. For example, the user may move the first bracket as described above to a location along the slider bar that corresponds to the fortieth image frame (e.g., image frame number 40) of the medical image dataset and may release (e.g., deselect) the first bracket at the location corresponding to the fortieth image frame in order to define the fortieth image frame as the beginning key frame of the image subset.

Selecting the end key frame may include selecting and moving a second bracket (e.g., second bracket 216 described above) from an opposing, second end of the slider bar to a desired end image frame of the image subset. As the user moves the second bracket along the slider bar, a frame number corresponding to the position of the second bracket along the slider bar may be displayed by the GUI for each of the different locations, and the user may release the second bracket at the desired location in order to define the end key frame (e.g., define the image frame of the medical image dataset that forms the end key frame of the image subset). The end key frame is an image frame that is selected from the plurality of image frames of the medical image dataset (e.g., the total amount of image frames of the medical image dataset) as described above. For example, the user may move the second bracket as described above to a location along the slider bar that corresponds to the sixtieth image frame (e.g., image frame number 60) of the medical image dataset and may release (e.g., deselect) the second bracket at the location corresponding to the sixtieth image frame in order to define the sixtieth image frame as the end key frame of the image subset.

Generating the image subset may further include, at 708, forming the image subset from the beginning key frame, the end key frame, and each image frame of the medical image dataset between the beginning key frame and the end key frame. For example, during conditions in which one or more image frames are arranged sequentially between the image frame of the medical image dataset defined as the beginning key frame of the image subset and the image frame of the medical image dataset defined as the end key frame of the image subset, each image frame between the beginning key frame and the end key frame is included by the image subset.

According to the example described above with reference to 706, the fortieth image frame of the medical image dataset may be selected as the beginning key frame of the image subset, and the sixtieth image frame of the medical image dataset may be selected as the end key frame of the image subset. During these conditions, the image subset is generated to include the fortieth image frame of the medical image dataset as the beginning key frame and the sixtieth image frame of the medical image dataset as the end key frame, and the forty-first image frame, the forty-second image frame, the forty-third image frame, and each other image frame of the medical image dataset arranged sequentially between the fortieth image and the sixtieth image frame is additionally included by the image subset. In this example, the image subset includes the fortieth image frame of the medical image dataset, the sixtieth image frame of the medical image dataset, and each image frame of the medical image dataset therebetween. Although the fortieth image frame and sixtieth image frame are provided as examples of the beginning key frame and end key frame, in other examples the beginning key frame and end key frame may correspond to different image frames of the medical image dataset (e.g., with the beginning key frame being the twentieth image frame and the end key frame being the thirtieth image frame, with the beginning key frame being the ninetieth image frame and the end key frame being the one hundred and thirtieth image frame, etc.).

At 712, the image subset is sorted to a visual indicator of the GUI and the visual indicator is displayed at the display device via the GUI. The visual indicator may be a tab, at least in some examples. For example, as described above, during conditions in which the image subset is generated by the medical image management system responsive to user input (e.g., defining the beginning key frame and end key frame as described above), the medical image management system further generates a visual indicator, such as a tab, associated with the image subset and displays the tab within the GUI (e.g., similar to tab 302 described above with reference to FIG. 2). The GUI may include a plurality of different tabs, with a main tab (e.g., similar to main tab 202 shown by FIG. 2 and described above) being associated with the entire medical image dataset, and with each other tab (e.g., tab 302, tab 502, tab 504, etc. shown by FIG. 5 and described above) being associated with a corresponding image subset generated by the medical image management system.

Sorting the image subset to the tab of the GUI and displaying the tab at the display device via the GUI may include, at 714, displaying image frames of the image subset at the display device via the GUI. For example, during conditions in which the tab is selected by the user (e.g., selected via input from the user interface device), image frames included by the image subset may be displayed at a display panel of the GUI (e.g., displayed at viewport 234 of display panel 232 described above with reference to FIGS. 2-6). A highlight portion of the tab (e.g., similar to highlight portion 303 described above with reference to FIG. 3) may include a fill (e.g., color or pattern fill) to indicate that the tab is selected. The display panel may display image frames of the image subset individually while the tab is selected (e.g., one at a time), or in some examples the display panel may display the image frames of the image subset as an animation (e.g., a sequence, as described above). In yet other examples, the display panel may display multiple image frames of the image subset together (e.g., side-by-side). During conditions in which the tab is not selected, the image frames of the image subset may not be shown at the display panel of the GUI, and the highlight portion may not include the fill. In this configuration, the user may select the tab in order to view the image frames of the image subset associated with the tab, and the user may deselect the tab (e.g., select a different tab, such as the main tab associated with the entire medical image dataset as described above) in order to hide the image frames of the image subset associated with the tab. In this way, the user may selectively view the image subset as desired by selecting the tab, or the user may hide the image subset by deselecting the tab. As a result, the user may more easily view the image frames of the medical image dataset included by the image subset without viewing an entirety of the medical image dataset, and productivity may be increased.

At 718, the image subset may optionally be output to a storage device or network. For example, the image frames included by the image subset may be saved to storage media (e.g., a hard drive) of the medical image management system for later use by the user (e.g., for repeated viewing, for display during patient consultation, etc.). The image frames included by the image subset may additionally or alternately be output (e.g., transmitted) to a network (e.g., network 105 shown by FIG. 1 and described above) for viewing at a different location (e.g., at a display screen located separately from the medical image management system, such as at a different office, building, etc.).

By configuring the medical image management system according to the examples described above, the medical image management system may generate a plurality of image subsets responsive to user input, and the user may navigate between the image subsets via the tabs displayed by the GUI. The respective label of each respective tab may be customized by the user to further distinguish each tab from each other tab. As a result, the user may more quickly and easily view desired portions of interest of the medical image dataset, and productivity may be increased.

The technical effect of generating an image subset from the medical image dataset and sorting the image subset to a respective tab of the GUI is to display the image subset separately from the medical image dataset and increase an ease of navigation of the GUI.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for a medical image management system, comprising:
acquiring a medical image dataset and displaying a timeline bar of the medical image dataset via a graphical user interface (GUI) of a display device;
adjusting a position of a first key image indicator and a position of a second key image indicator along the timeline bar;
generating an image subset from the medical image dataset based on the position of the first key image indicator and the position of the second key image indicator and storing the image subset for replay; and
forming a visual indicator of the image subset within the GUI that when selected, displays the image subset.

2. The method of claim 1, wherein generating the image subset from the medical image dataset based on the position of the first key image indicator and the position of the second key image indicator includes:
determining a first key image of the image subset from the medical image dataset based on the position of the first key image indicator; and
determining a second key image of the image subset from the medical image dataset based on the position of the second key image indicator.

3. The method of claim 2, wherein the first key image defines a beginning boundary of the image subset and the second key image defines an end boundary of the image subset.

4. The method of claim 3, wherein forming the visual indicator of the image subset includes generating a label of the visual indicator from a frame number of the first key image and a frame number of the second key image.

5. The method of claim 4, wherein the visual indicator of the image subset comprises a tab.

6. The method of claim 4, further comprising:
adjusting a position of a third key image indicator and a position of a fourth key image indicator along the timeline bar;
generating a second image subset from the medical image dataset based on the position of the third key image indicator and the position of the fourth key image indicator and storing the second image subset for replay; and
forming a second visual indicator of the second image subset within the GUI that when selected, displays the second image subset.

7. The method of claim 6, wherein generating the second image subset from the medical image dataset based on the position of the third key image indicator and the position of the fourth key image indicator includes:
determining a third key image defining a beginning boundary of the second image subset from the medical image dataset based on the position of the third key image indicator; and
determining a fourth key image defining an end boundary of the second image subset from the medical image dataset based on the position of the fourth key image indicator.

8. The method of claim 7, wherein a frame number of the third key image or a frame number of the fourth key image is between the frame number of the first key image and the frame number of the second key image.

9. The method of claim 4, further comprising:
selecting the visual indicator within the GUI; and
responsive to selecting the visual indicator, displaying the image subset as a replayable animation at a display panel of the GUI.

10. The method of claim 9, further comprising:
responsive to selecting the visual indicator within the GUI, displaying the frame number of the first key image at a first frame number indicator, and displaying the frame number of the second key image at a second frame number indicator.

11. The method of claim 9, further comprising:
responsive to selecting the visual indicator, displaying a fill at a highlight portion of the visual indicator.

12. The method of claim 9, wherein the selecting of the visual indicator within the GUI occurs automatically responsive to the forming of the visual indicator.

13. The method of claim 1, wherein adjusting the position of the first key image indicator includes moving the first key image indicator to a first position along the timeline bar, where the first position is one of a plurality of different positions along the timeline bar, with each position of the plurality of different positions representing a respective image frame of the medical image dataset.

14. A medical image management system, comprising:
a display device;
a processor;
a user interface device; and
a non-transitory memory configured with executable instructions stored thereon that when executed cause the processor to:
acquire, from a medical imaging system, a medical image dataset and display a timeline bar of the medical image dataset via a graphical user interface (GUI) of the display device;
generate an image subset from the medical image dataset responsive to a user input to the GUI via the user interface device; and
form a visual indicator of the image subset within the GUI, automatically select the visual indicator, and display the image subset within the GUI while the visual indicator is selected.

15. The medical image management system of claim 14, further comprising executable instructions stored on the non-transitory memory that when executed, cause the processor to:
display the medical image dataset at a display panel of the GUI prior to the generation of the image subset; and
responsive to the forming of the visual indicator of the image subset, replace the medical image dataset at the display panel with the image subset.

16. The medical image management system of claim 14, wherein the timeline bar includes a first end representing a beginning image frame of the medical image dataset and a second end representing an end image frame of the medical image dataset, with a beginning key frame of the image subset represented by a first bracket on the timeline bar and an end key frame of the image subset represented by a second bracket on the timeline bar.

17. The medical image management system of claim 16, further comprising executable instructions stored on the non-transitory memory that when executed, cause the processor to:
adjust a position of the first bracket on the timeline bar responsive to a user input to move the first bracket via the user interface device;
adjust a position of the second bracket on the timeline bar responsive to a user input to move the second bracket via the user interface device; and
update the beginning key frame of the image subset based on the adjusted position of the first bracket, and update the end key frame of the image subset based on the adjusted position of the second bracket.

18. A method, comprising:
acquiring a medical image dataset and displaying the medical image dataset at a display panel of a graphical user interface of a display device;
generating a first image subset from the medical image dataset by selecting a beginning, first key frame and an end, second key frame of the first image subset from a total amount of image frames of the medical image dataset responsive to a user input; and
while generating the first image subset:
generating a first label for the first image subset from a frame number of the first key frame and a frame number of the second key frame;
displaying the first label at a first tab of the graphical user interface; and
clearing the display panel, then displaying the first image subset at the display panel.

19. The method of claim 18, further comprising:
generating a second image subset from the medical image dataset by selecting a beginning, third key frame and an end, fourth key frame of the second image subset from the plurality of image frames of the medical image dataset responsive to a user input, where at least a portion of the second image subset overlaps at least a portion of the first image subset;
while generating the second image subset:
generating a second label for the second image subset from a frame number of the third key frame and a frame number of the fourth key frame;
displaying the second label at a second tab of the graphical user interface; and
responsive to a selection of the second tab, clearing the display panel, then displaying the second image subset at the display panel.

20. The method of claim 19, wherein the first image subset includes a larger, first range of image frames of the medical image dataset, the second image subset includes a smaller, second range of image frames of the medical image dataset, and the second range of image frames of the medical imaging dataset is nested within the first range of image frames of the medical imaging dataset.
